(19) United States
(12) Patent Application Publication
(10) Pub. No.: US 2025/0000462 A1
(43) Pub. Date: Jan. 2, 2025

(54) INFORMATION PROCESSING APPARATUS, INFORMATION PROCESSING METHOD, INFORMATION PROCESSING SYSTEM, AND STORAGE MEDIUM

(71) Applicant: CANON KABUSHIKI KAISHA, Tokyo (JP)

(72) Inventor: YOSHIHIKO SUGAWA, Saitama (JP)

(21) Appl. No.: 18/733,995

(22) Filed: Jun. 5, 2024

(30) Foreign Application Priority Data
Jun. 27, 2023 (JP) 2023-105286

(51) Int. Cl. A61B 5/00; A61B 5/021; A61B 5/024; A61B 5/08; A61B 5/1455; G06T 7/20; G06V 10/25; G06V 40/10

(52) U.S. Cl. CPC A61B 5/7285; A61B 5/021; A61B 5/02416; A61B 5/0816; A61B 5/14551; G06T 7/20; G06V 10/25; G06V 40/10; G06T 2207/10016; G06T 2207/30201

(57) ABSTRACT

An information processing apparatus includes a memory storing instructions, and a processor configured to execute the instructions to acquire a moving image of a target to be inspected, from an image pickup apparatus, detect a plurality of body regions of the target to be inspected, detect event information regarding a change in an object in the moving image, set one body region selected from the plurality of body regions based on the event information, and acquire biological information on the target to be inspected, from the set body region.

### BACKGROUND

### Technical Field

One of the aspects of the embodiments relates to an information processing apparatus, an information processing method, an information processing system, and a storage medium.

### Description of Related Art

Noncontact biological (or biometric) information detecting technologies have recently been proposed. The principle of acquiring biological information such as a respiration rate and a pulse rate from a moving image acquired by an image pickup apparatus is similar to photoplethysmography (PPG). A light absorption characteristic of hemoglobin in blood is used to detect biological information. Since the green component is well absorbed in subcutaneous blood vessels, the signal intensity of the green component of reflected light changes depending on the increase or decrease of blood in the blood vessel. Therefore, the biological information detecting apparatus is configured to detect time-series data (PPG signal) containing biological information using light with a wavelength in which hemoglobin has a high light absorption characteristic. That is, in order to detect biological information, a target region is to be set in a region including a skin region. In addition to information regarding a pulse wave, the PPG signal may further include information due to physiological phenomena, such as respiration. Then, for example, the respiration rate and pulse rate can be calculated from peak intervals of the detected time-series PPG signals. Acquiring biological information on a target to be inspected in a noncontact manner can provide a state of the target to be inspected while the stress on the target to be inspected is minimized. For example, it can be used to monitor a patient in a hospital and to recognize his condition in an examination room and treatment room.

The noncontact biological information detecting technology for a target to be inspected contact may deteriorate the biological information detecting accuracy due to the motion of the target to be inspected, for example, due to the motion of the face of the target to be inspected. Japanese Patent Laid-Open No. 2022-045503 discloses a target area selecting method using the distribution (variance) of time-series data acquired by an image pickup apparatus.

However, in acquiring biological information on a target to be inspected in a noncontact manner, the motion of a face of the target to be inspected is not the only factor that degrades the detecting accuracy of biological information. In a case where the skin region of the target to be inspected becomes a blind spot due to an event that overlaps the skin region of the target to be inspected, biological information on the target to be inspected cannot be acquired. Here, an event is a motion of an object that affects the skin region of the target to be inspected who is to be imaged. For example, in an X-ray room or treatment room in a hospital, the skin region of the target to be inspected becomes a blind spot depending on the motion of a detecting apparatus or the motion of an object such as a doctor, a nurse, and a radiographer and thus the biological information on a patient cannot be continuously monitored.

The biological information detecting apparatus disclosed in Japanese Patent Laid-Open No. 2022-045503 selects a target area of a target to be inspected only by the time-series distribution data including the facial motion of the target to be inspected. Therefore, this biological information detecting apparatus may not continuously detect biological information in a case where the skin region of the target to be inspected becomes a blind spot due to the motion of the target to be inspected, the motions of the apparatus and/or a person near the target to be inspected, and the like.

### SUMMARY

An information processing apparatus according to one aspect of the disclosure includes a memory storing instructions, and a processor configured to execute the instructions to acquire a moving image of a target to be inspected, detect a plurality of body regions of the target to be inspected, from the moving image, detect event information regarding a change in an object in the moving image, set one body region selected from the plurality of body regions based on the event information, and acquire biological information on the target to be inspected, from the set body region. An information processing system including the above information processing apparatus also constitutes another aspect of the disclosure. An information processing method corresponding to the above information processing apparatus also constitutes another aspect of the disclosure. A storage medium storing a program that causes a computer to execute the above information processing method also constitutes another aspect of the disclosure.

Further features of various embodiments of the disclosure will become apparent from the following description of embodiments with reference to the attached drawings.

### BRIEF DESCRIPTION OF THE DRAWINGS

FIG. 1 is a block diagram of detecting systems according to Examples 1 to 3.

FIGS. 2A to 2C explain a frequency characteristic in each example.

FIG. 3 is a flowchart illustrating detecting processing according to Examples 1 and 2.

FIGS. 4A to 4C explain the detecting processing according to Example 1.

FIGS. 5A and 5B explain time-series data in Example 2.

FIG. 6 is a flowchart illustrating detecting processing according to Example 3.

FIG. 7 is a block diagram of a detecting system according to Example 4.

FIG. 8 is a flowchart illustrating detecting processing according to Example 4.

### DESCRIPTION OF THE EMBODIMENTS

In the following, the term “unit” may refer to a software context, a hardware context, or a combination of software and hardware contexts. In the software context, the term “unit” refers to a functionality, an application, a software module, a function, a routine, a set of instructions, or a program that can be executed by a programmable processor such as a microprocessor, a central processing unit (CPU), or a specially designed programmable device or controller. A memory contains instructions or programs that, when executed by the CPU, cause the CPU to perform operations corresponding to units or functions. In the hardware context, the term “unit” refers to a hardware element, a circuit, an assembly, a physical structure, a system, a module, or a subsystem. Depending on the specific embodiment, the term “unit” may include mechanical, optical, or electrical components, or any combination of them. The term “unit” may include active (e.g., transistors) or passive (e.g., capacitor) components. The term “unit” may include semiconductor devices having a substrate and other layers of materials having various concentrations of conductivity. It may include a CPU or a programmable processor that can execute a program stored in a memory to perform specified functions. The term “unit” may include logic elements (e.g., AND, OR) implemented by transistor circuits or any other switching circuits. In the combination of software and hardware contexts, the term “unit” or “circuit” refers to any combination of the software and hardware contexts as described above. In addition, the term “element,” “assembly,” “component,” or “device” may also refer to “circuit” with or without integration with packaging materials.

Referring now to the accompanying drawings, a detailed description will be given of embodiments according to the disclosure. Each drawing may have a scale different from the actual scale for convenience. Corresponding elements in respective figures will be designated by the same reference numerals, and a duplicate description thereof will be omitted. The following examples do not limit this disclosure. Although a plurality of features are described in each embodiment, not all of these features are essential, and the plurality of features may be arbitrarily combined.

It is generally known that biological information can be detected by irradiating light onto a part of a living body that is a target to be inspected and by detecting a reflected light amount from the living body. It is also known that hemoglobin in blood absorbs green visible light with a wavelength band of 495 to 570 nm. A G (green) signal of a color filter in an image pickup apparatus includes many signals that pass through the wavelength band of 495 to 570 nm and can improve the measuring accuracy of the respiration rate and heart rate.

More specifically, capillaries exist on the skin surface, and in a case where the blood flow through the blood vessels changes due to the heartbeat, a light amount absorbed by the bloodstream also changes according to the heartbeat and a luminance change obtained by reflection from the skin region also changes with the heartbeat. Although a luminance change amount is small, in a case where the average luminance value within the target region is calculated, time-series data of the luminance include a biological information signal component. Accordingly, for example, by using time-series data of each signal having a different light amount absorption by hemoglobin in the blood in which among three signals, that is, a G signal, an R (red) signal, and a B (blue) signal, the biological information signal is detected. That is, in order to detect a biological information signal, a region including a skin region is to be set as a target region.

In detecting biological information in a noncontact manner, the biological information signal intensity contained in the reflected light changes according to the motion of the target to be inspected, the motion of an apparatus near the target to be inspected, and the motion of a person near the target to be inspected, in addition to periodic light intensity changes due to the biological information on the target to be inspected. In a case where the motion of the target to be inspected, the motion of the apparatus near the target to be inspected, or the motion of the people near the target to be inspected does not occur, the biological information signal intensity change included in the skin region of the target to be inspected is small. On the other hand, in a case where the motion of the target to be inspected, the motion of an apparatus near the target to be inspected, or the motion of a person near the target to be inspected occurs, the biological information signal intensity change included in the skin region of the target to be inspected is large.

The motion of a target to be inspected includes, for example, a motion of changing a direction of a target to be inspected in accordance with an instruction from a doctor, nurse, radiographer, etc. during a barium examination in a hospital. Depending on the motion of changing the direction of the target to be inspected, a part of a skin region of the target to be inspected becomes a blind spot, and the biological information on the target to be inspected cannot be continuously monitored.

The motion of apparatus near the target to be inspected includes, for example, a motion of a detecting apparatus etc. in an examination room or a patient room in a hospital. Each example can be widely applied to various apparatuses such as an X-ray TV examination apparatus and a portable X-ray image pickup apparatus, and the applicable apparatuses are not limited. Due to the operation of the detecting apparatus or the like, the intensity of the detected biological information signal decreases as the skin region of the target to be inspected overlaps the apparatus. In other words, it becomes impossible to continuously monitor biological information.

The motion of a person near the target to be inspected includes, for example, a motion of a medical staff (or worker) in an examination room or treatment room in a hospital. The medical staff includes a doctor, nurse, radiographer, etc., and the medical staff is not limited in each example. Due to the motion of the medical staff, the skin region of the target to be inspected overlaps the medical staff, and the biological information signal intensity decreases. In other words, highly accurate biological information cannot be continuously monitored.

In a case where an illumination change occurs, the biological information signal intensity included in the body region of the target to be inspected changes significantly. The illumination change includes, for example, a change in the illumination environment caused by a medical staff illuminating a part of the skin region of a target to be inspected in an examination room in a hospital. Since the conditions for extracting the skin region change due to the illumination change, the skin region range of the target to be inspected reduces and the biological information signal intensity reduces. In other words, biological information cannot be continuously monitored.

In a case where the motion of the target to be inspected, the motion of an apparatus near the target to be inspected, or the motion of a person near the target to be inspected is detected, biological information can be continuously detected with high accuracy by setting a target region to a body region including a skin region where the biological information signal intensity is high. The body region includes a skin region such as a face region including facial parts such as the eyes, nose, mouth, forehead, cheeks, and facial contours of the target to be inspected, a hand region and a foot region. The face region is not limited to the entire face defined based on feature points of the face region type such as the eyes, nose, mouth, and facial contour, but may be a specific part range such as the cheeks and forehead. The hand region is not limited to the entire hand or arm, but may be a specific region range such as the palm, wrist, or forearm. The foot region is not limited to the foot region, but may be a specific region such as the footpad.

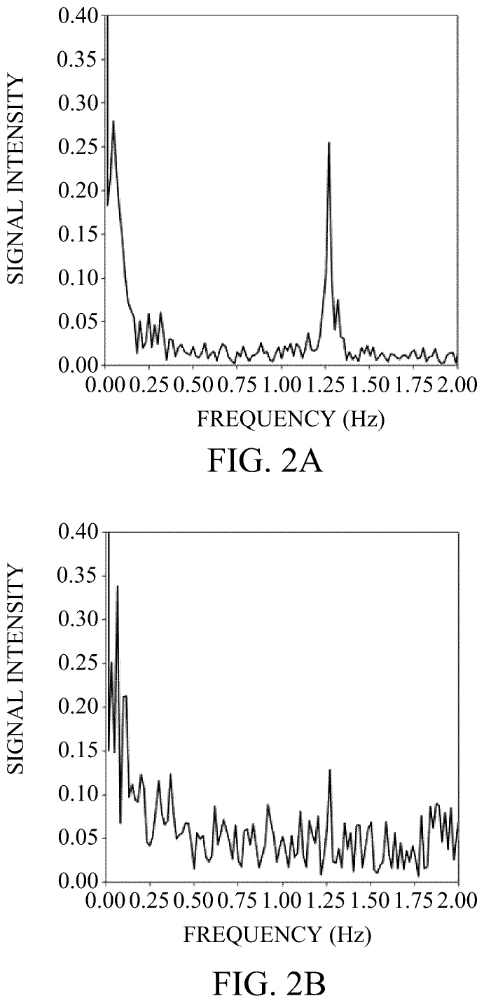

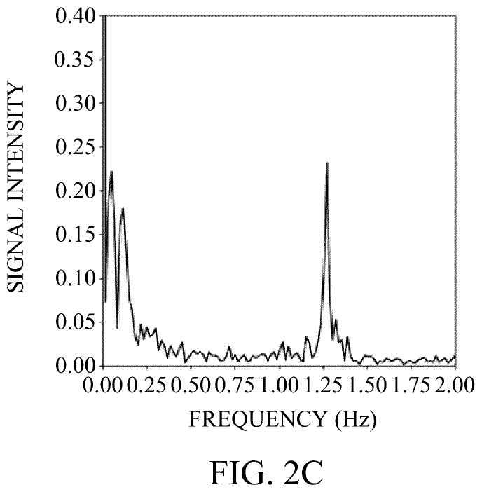

FIGS. 2A to 2C illustrate a frequency characteristic result obtained by Fourier-transforming time-series data of an average luminance value signal of the G signal for 60 seconds in a case where a moving image of a target to be inspected with a pulse rate of approximately 75 bpm is acquired and a target region is set to a skin region of a face region or a hand region. The peak frequency of the biological information signal of the pulse component of the target to be inspected this time is 1.25 Hz. In FIGS. 2A to 2C, the vertical axis represents the biological information signal intensity (signal intensity), and the horizontal axis represents the frequency (Hz). FIG. 2A illustrates frequency information obtained by frequency analysis of a biological information signal within a face region of a target to be inspected. FIG. 2B illustrates frequency information obtained from a biological information signal acquired by setting the face of the target to be inspected as a target region in a state where most of the face region of the target to be inspected overlaps the motion of an apparatus near the target to be inspected. FIG. 2C illustrates frequency information obtained from a biological information signal acquired by setting the hand of the target to be inspected as a target region in a state where most of the face region of the target to be inspected overlaps the motion of a thing near the target to be inspected.

As illustrated in FIGS. 2A to 2C, in a case where the body region of the target region becomes a blind spot due to the motion of the apparatus near the target to be inspected, the detected biological information signal intensities differ. When FIGS. 2A and 2B are compared, it can be understood that FIG. 2A has a frequency at which the signal intensity peaks at 0.25 around 1.25 Hz. It can be understood that FIG. 2B does not have a peak frequency around 1.25 Hz. In other words, it can be understood that the pulse component, which is one type of biological information, cannot be detected because the body region information decreases due to the motion near the target to be inspected. When FIGS. 2B and 2C are compared, it can be understood that FIG. 2C has a frequency at which the signal intensity peaks at 0.23 around 1.25 Hz. In other words, it can be understood that the pulse component, which is one type of biological information, can be continuously detected by switching the body region based on the motion of the apparatus near the target to be inspected.

However, a general biological information detecting apparatus detects biological information from a preset target region without changing the target region set to the body region of the target to be inspected. Accordingly, event information (on an object change in a moving image) that includes the motion of the target to be inspected in the moving image acquired by the image pickup apparatus, the motion of an apparatus near the target to be inspected, the motion of a person near the target to be inspected, etc.

Here, the event is a motion (or action) that affects a skin region (body region) to be imaged of a target to be inspected due to the motion of the object. The object includes, but is limited to, a target to be inspected in a moving image, a person different from the target to be inspected (such as a medical staff performing a procedure), and an object or thing (an apparatus in an examination room). Further, the event may be a illumination change.

The event information refers to information from a moving image for detecting the motion of an object (information regarding the motion of an object). In acquiring event information from a moving image, a movement of a detecting apparatus in a specific area or a motion of a body region of a target to be inspected may be detected by block matching or the like. Further, human actions performed for a target to be inspected, such as an action, e.g., a treatment performed by a doctor, nurse, radiographer, etc., for a target to be inspected may be acquired as event information. In a case where an object is included in a moving image in acquiring event information as the motion of the object from the moving image, the event information may be acquired as event information such as a treatment or the event information may be acquired as event information such as a treatment by detecting the motion of the target to be inspected. Alternatively, the event information may be acquired as event information such as a treatment by detecting a specific motion of an object that provides the specific motion. However, each example is not limited to this implementation as long as the event information can be acquired from a moving image. Furthermore, the event information may be set to an illumination change or whether it is bright or dark (information regarding an illumination change) based on a moving image. Biological information can be continuously detected with high accuracy by continuously switching the setting of a target region to a body region including a large amount of body region information based on whether or not the event information is detected.

Here, the body region information is the total number of pixels acquired from each body region or the amplitude change of time-series data consisting of the total number of pixels. The total number of pixels may be set to a region including the body region, and pixels included in a skin region expressed in a color space component such as the HSV color space. Thereby, the biological information signal intensity can be increased by evaluating only the pixels that include the biological information signal.

### Detecting System

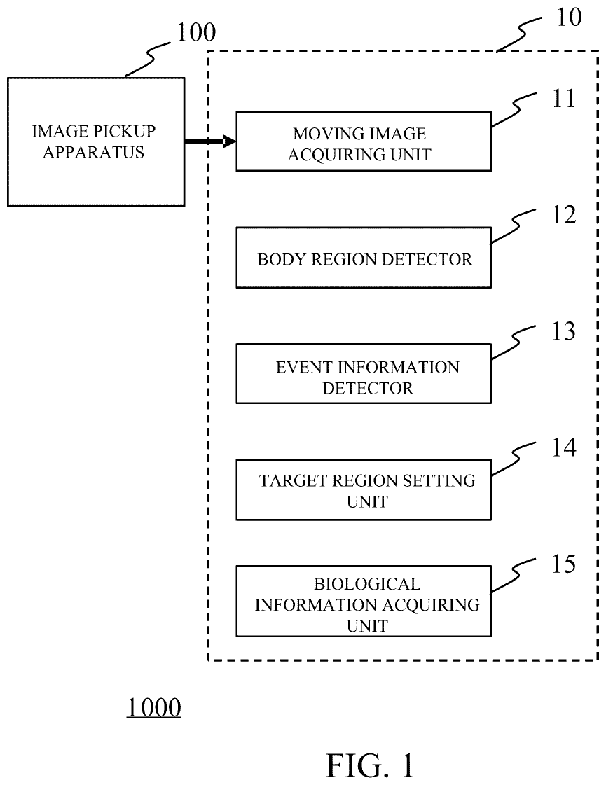

Referring now to FIG. 1, a description will be given of a detecting system (information processing system) 1000 including a biological information detecting apparatus (information processing apparatus) 10 according to each example. FIG. 1 is a block diagram of the detecting system 1000. The detecting system 1000 is a system configured to detect biological information by temporally continuously acquiring information regarding reflected light from a skin surface of a target to be inspected from a moving image obtained by imaging the skin surface of the target to be inspected using the image pickup apparatus 100. The detecting system 1000 includes an image pickup apparatus 100 and a biological information detecting apparatus 10. The biological information detecting apparatus 10 and the image pickup apparatus 100 are communicably connected to each other. The connection method may be wired or wireless. In the detecting system 1000, the biological information detecting apparatus 10 and the image pickup apparatus 100 may be configured as an integrated apparatus. That is, the image pickup apparatus 100 may be provided as an imaging unit inside the biological information detecting apparatus 10, or the biological information detecting apparatus 10 may be provided as an inspecting unit inside the image pickup apparatus 100.

The image pickup apparatus 100 can capture a target to be inspected at 30 fps (frames per second). In other words, a moving image can be obtained by continuously recording 30 images per second. The frame rate of the image pickup apparatus 100 is not limited to 30 fps. The image pickup apparatus 100 includes an optical system (imaging optical system) and an image sensor. The image sensor includes pixels that are sensitive to at least one wavelength. At least one of the pixels described above is a pixel that is sensitive to a biological information component. The image sensor may have pixels that are sensitive to two or more types of wavelengths, such as an image sensor that has color filters for three colors of RGB. The image sensor can use a photoelectric conversion element such as a CCD sensor or a CMOS sensor.

The biological information detecting apparatus 10 includes a moving image acquiring unit 11, a body region detector (region detector) 12, an event information detector (information detector) 13, a target region setting unit (region setting unit) 14, and a biological information acquiring unit (information acquiring unit) 15. In such a configuration, the biological information detecting apparatus 10 inspects the target to be inspected, which is imaged by the image pickup apparatus 100. The program that execute various processing performed by the biological information detecting apparatus 10 according to each example can also be realized by processing in which one or more processors in the computer of the system or apparatus read and execute the program via a network or storage medium. The program can also be realized by processing in which one or more processors in the computer of the system or apparatus read the program and execute the various processing performed by the biological information detecting apparatus 10.

The moving image acquiring unit 11 acquires a moving image of the target to be inspected from the image pickup apparatus 100, that is, acquires a luminance value output from the image sensor in the image pickup apparatus 100. In each example, since the image pickup apparatus 100 temporally continuously images the target to be inspected, the moving image acquiring unit 11 can also acquire luminance values corresponding to temporally continuous images. In that case, the moving image acquiring unit 11 may acquire information regarding time. The information regarding time is a period from the imaging start or the number of images (number of frames) captured since the imaging start. Such a configuration can detect biological information on a target to be inspected at a specific timing during imaging.

The body region detector 12 detects at least two body regions in each frame image received from the moving image acquiring unit 11.

The event information detector 13 acquires event information that is an event relating to the motion of the object. Here, the event information is information regarding the motion of the object. The motion of the object is the motion of the target to be inspected, the motion of an apparatus near the target to be inspected, and the motion of a person near the target to be inspected.

The target region setting unit 14 sets a target region by evaluating the body region detected by the body region detector 12 (sets one body region selected from the plurality of regions) based on a result of the event information detector 13 (event information regarding an object change). The target region setting unit 14 sets arbitrary size and shape ranges, including one body region, as a target region.

A skin region that includes a region range including a facial part and corresponds to a skin range expressed by a color space component such as HSV color space may be set as the target region. Thereby, the biological information detecting accuracy can be improved. Based on the result of the event information detector 13, the target region can be switched by comparing the total number of pixels, which is body region information obtained from each of the body regions detected by the body region detector 12. The body region with the largest total number of pixels is set as the target region.

As an example, a case will be described using FIGS. 2A and 2C. From FIG. 2A, the signal intensity at the peak frequency is 0.25. From FIG. 2C, the signal intensity at the peak frequency is 0.23. A ratio of the total number of pixels in the body regions in FIGS. 2A and 2C is 12:1. In other words, as the total number of pixels included in the body region increases, the signal intensity becomes higher. That is, by setting the body region with the largest total number of pixels as the target region, the pulse rate, which is biological information, can be acquired. Since only the total number of pixels is evaluated, calculation processing is fast and target region can be quickly switched. The total number of pixels may be set to be a region range including the body region, and pixels corresponding to a skin region expressed by a color space component such as HSV color space. Thereby, the selecting accuracy of a body region with high signal intensity can be improved by evaluating only the number of pixels containing biological information. Therefore, the biological information detecting accuracy can be improved.

Furthermore, by switching the target region based on the result of the event information, the body region is selected from the moving image obtained through imaging and biological information is detected, and thus the detecting accuracy can be improved.

The biological information acquiring unit 15 acquires biological information on the target to be inspected from one body region (target region) set by the target region setting unit 14. That is, the biological information detecting apparatus 10 detects biological information on the target to be inspected from the signal corresponding to the target region set by the target region setting unit 14.

The signal is an average luminance value extracted from a channel passing through a color filter in a solid-state image sensor within the image pickup apparatus 100. For example, a signal of a channel having spectral sensitivity in the red wavelength band is R, a signal of a channel having spectral sensitivity in the green wavelength band is G, and a signal of a channel having spectral sensitivity in the blue wavelength band is B. The average luminance value is calculated from luminance values included within the target region of one frame image. By detecting biological information using the average luminance value, the biological information detecting accuracy can be increased by reducing the noise influence.

Finally, time-series data is obtained by extracting a signal for each frame image. The time-series data is not limited to the average luminance value of the G signal, but may be the average luminance value of the R signal or the average luminance value of the B signal. For example, in generating time-series data using only the G signal, the average luminance value of the G signal is extracted in time series and moving average processing is performed. Further, the time-series data is not limited to the average luminance value of the signal of a single channel, but may be a value calculated by four arithmetic operations of signals of a plurality of channels. In the following, the average luminance value corresponding to the G pixel will be referred to as Gave, the average luminance value corresponding to the R pixel will be referred to as Rave, and the average luminance value corresponding to the B pixel will be referred to as Bave. In a case where two types of signals, G and R signals, are used, it can be expressed by Gave/Rave.

Although the case where two signals, the G and R signals, are used as the signal selection is illustrated, as long as there are a plurality of different signals, any number of signals can be selected. For example, any combination of signals that transmit light in wavelength bands such as a G signal, an R signal, a B signal, an IR (infrared) signal, etc. can be used; two or more signals may be used. Since IR has a deep penetration depth into the living body, it contains information on deep blood flow. In other words, biological information can be detected even in a dark field environment that is invisible to the human eyes. Thereby, the biological information detecting accuracy at night can be improved. The time-series data generating processing may include moving average processing and other noise removal processing. Thereby, time-series data changes can be smoothed and detection of a waveform peak position can be facilitated.

A peak (maximum value or minimum value) is detected from the generated time-series data, and a pulse value is calculated and output as biological information based on the frame rate of the image pickup apparatus 100. The frame rate represents the number of frames captured per unit time. For example, in a case where the frame rate of the image pickup apparatus 100 is 30 fps (frames per second) and the interval between two adjacent peaks (difference in frame numbers) is 30, the pulse rate becomes 60×30÷30=60 times/min. The pulse rate may be calculated from the time average of two adjacent peak intervals, the average peak interval calculated from the two adjacent peak intervals, or the number of peaks within a specific time. Alternatively, the respiration rate is calculated by obtaining peak positions by applying a bandpass filter to the time-series data to extract a band in which the respiration rate can exist, and by calculating the peak interval or the number of peaks within a certain time range.

Generating a waveform as time-series data that includes biological information from which noise components including motion components have been removed can accurately capture a feature amount of the waveform. For example, a peak interval change or a amplitude change in the generated waveform can be used as an index to indicate whether or not the pulse is in an arrhythmia state. The method for acquiring the waveform feature amount is not limited to the method described above, and may use any method.

For example, biological information may be acquired from a second-order differential of the generated waveform, or from a feature amount (inflection point) that is not known in the originally generated waveform. Furthermore, the acquired feature amount may be used to discover an early disease.

Thus, generating waveforms as time-series data that include biological information from which noise components have been removed can enable early-stage diseases to be detected and highly accuracy vital measurement to be performed. Alternatively, the pulse rate and respiratory rate may be calculated from the peak frequency in the frequency characteristic obtained by Fourier-transforming the time-series data. Furthermore, peaks may be detected from the time-series data and pulse fluctuations may be calculated from temporal peak-interval fluctuations. Alternatively, a maximum value and a minimum value may be detected from the time-series data, and oxygen saturation fluctuations may be calculated from temporal width fluctuations.

The method of acquiring biological information includes a method of calculating a moving average of intervals between waveform peaks, a frequency analysis method, a method of analyzing a principal component, and the like, and the biological information acquiring method according to this embodiment is not limited. The biological information is acquired by pulse, respiratory rate, blood pressure, and oxygen saturation of a target to be inspected. Biological information to be acquired can be selected from time-series data to be acquired.

Each example will be described in detail below.

### Example 1

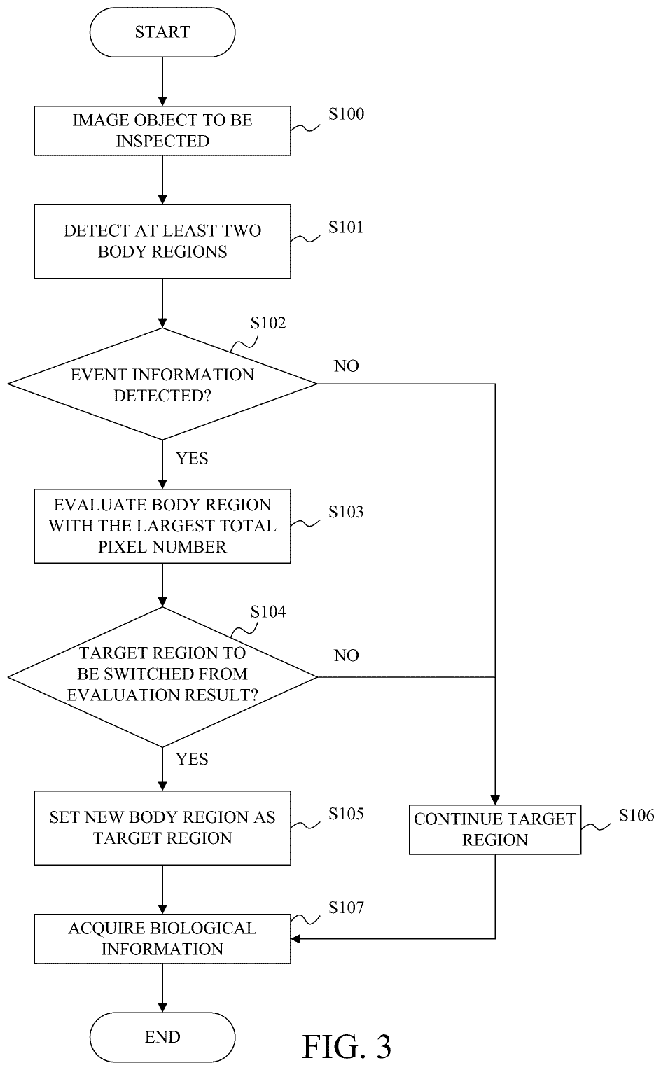

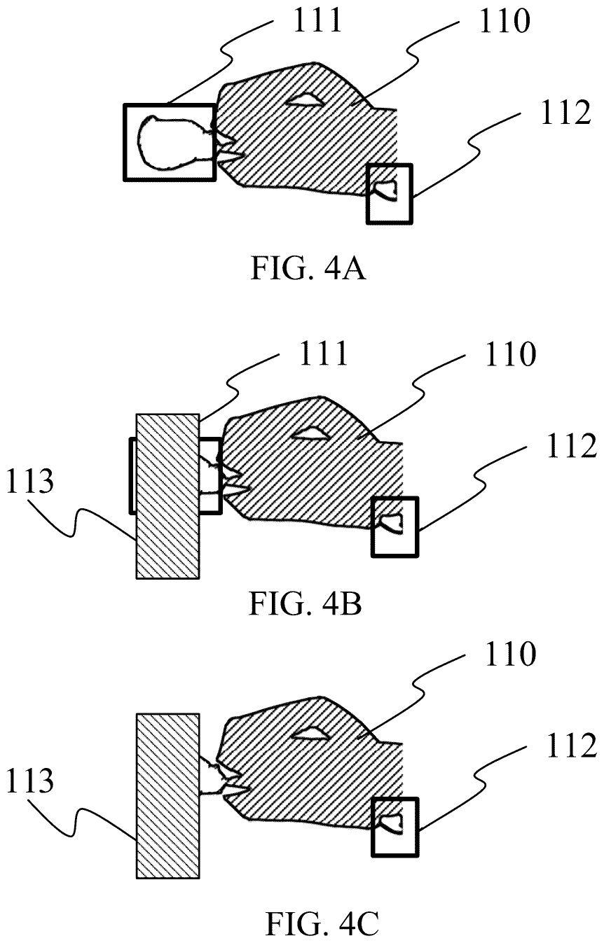

Referring now to FIGS. 3, 4A, 4B, and 4C, a description will be given of a detecting method (biological information detecting processing) by the biological information detecting apparatus 10 according to Example 1. FIG. 3 is a flowchart illustrating biological information detecting processing according to this example. FIGS. 4A to 4C explain biological information detecting processing, and illustrate a series of flows up to setting a target region.

In FIG. 4A, the body region detector 12 detects a face region 111 and a hand region 112 as body regions of a target to be inspected 110. In FIG. 4B, the event information detector 13 detects the motion of an object 113. The object 113 has moved to a position overlapping the face region 111. In FIG. 4C, the target region setting unit 14 sets the hand region 112 as the target region.

First, in step S100 (imaging step) in FIG. 3, the image pickup apparatus 100 images a target to be inspected. In this example, the image sensor included in the image pickup apparatus 100 is an RGB color sensor. The RGB color sensor has a G pixel that is sensitive to the G wavelength band, an R pixel that is sensitive to the R wavelength band, and a B pixel that is sensitive to the B wavelength band. The moving image acquiring unit 11 acquires a luminance value output from the image sensor. In this example, since the image sensor is an RGB color sensor, the moving image acquiring unit 11 acquires the G luminance value, the R luminance value, and the B luminance value.

Next, in step S101 (body region detecting step), the body region detector 12 detects at least two body regions (a plurality of body regions) of a face region including some of the facial parts such as the eyes, nose, mouth, cheeks, forehead, and facial outline of the target to be inspected, a hand region, and a foot region. For example, in FIG. 4A, the body region detector 12 detects the face region 111 and the hand region 112 as the body regions of the target to be inspected 110.

Next, in step S102 (event information detecting step), the event information detector 13 determines whether event information has been detected. The event information is information regarding the motion of the target to be inspected, the motion of an apparatus near the target to be inspected, the motion of a person near the target to be inspected, and the like.

In a case where the event information detector 13 determines that event information has been detected, the flow proceeds to step S103. For example, as illustrated in FIG. 4B, in a case where the object 113 has moved to a position overlapping the face region 111, the event information detector 13 detects the motion of the object 113 as event information. On the other hand, in a case where the event information detector 13 determines that no event information has been detected, the flow proceeds to step S106. In step S106, the target region setting unit 14 continues the current target region without switching the target region.

In step S103 (body region information evaluation step), the target region setting unit 14 compares (evaluates) the total pixel numbers, which are body region information acquired from each body region. In this example, the target region setting unit 14 sets the body region with the largest total pixel number as the target region. The target region setting unit 14 compares the total number of pixels in the face region 111 and the total number of pixels in the hand region 112, as illustrated in FIG. 4B, for example. Now assume that the total number of pixels in the hand region 112 is larger than the total number of pixels in the face region 111.

Next, in step S104 (target region switching step), the target region setting unit 14 determines whether to switch the target region based on the evaluation result (comparison result) of step S103. That is, the target region setting unit 14 evaluates event information regarding object changes and determines whether to switch one body region for acquiring biological information among the plurality of body regions. In a case where it is determined that the target region is to be switched, the flow proceeds to step S105.

On the other hand, in a case where it is determined that the target region is not to be switched, the flow proceeds to step S106.

In step S105 (target region setting step), the target region setting unit 14 sets a new body region as a target region. The target region setting unit 14 sets, for example, the hand region 112 illustrated in FIG. 4C as the target region.

Next, in step S107 (biological information acquisition step), the biological information acquiring unit 15 acquires biological information based on the target region set in step S105 or S106.

Thus, according to the biological information detecting apparatus 10 according to this example, in a case where event information is detected, the target region can be switched based on the total number of pixels acquired from each body region. Therefore, the accuracy of continuously acquired biological information can be improved.

### Example 2

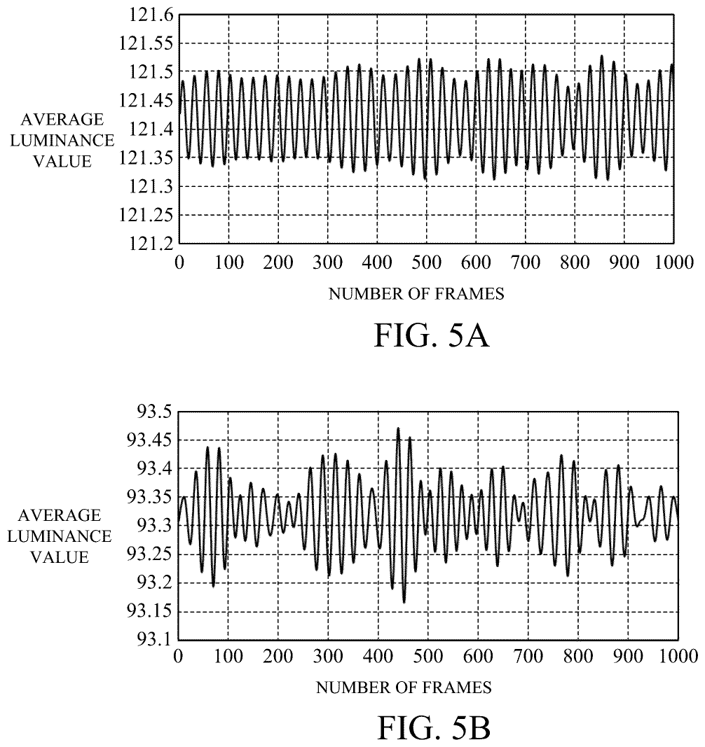

Referring now to FIGS. 3, 5A, and 5B, a description will be given of a biological information detecting apparatus (information processing apparatus) 10 according to Example 2. In this example, the configuration of the image pickup apparatus 100 is similar to that of Example 1, and thus a description thereof will be omitted.

In step S103 (body region information evaluating step) in FIG. 3, the target region setting unit 14 generates time-series data to be acquired, from each body region. An average luminance value is calculated from the luminance value output by the total number of pixels in a range corresponding to the body region set by the body region detector 12. Arranging average luminance values in time series can provide time-series data. Furthermore, the total number of pixels may be set to the number of pixels corresponding to the skin region.

In detecting time-series data using only a G signal, the average luminance value of the G signal is extracted as the time-series data. In detecting time-series data using a G signal and an R signal, the average luminance value of the G signal/the average luminance value of the R signal is extracted in time series. In detecting time-series data using a G signal, a B signal, and an R signal, the average luminance value of the G signal/(average luminance value of the R signal+average luminance value of the B signal) is extracted in time series. In order to emphasize the average luminance value of the G signal more than the average luminance values of the other signals, the average luminance value of the G signal, which is the numerator, may be squared as in Gave²/(Rave×Bave).

Thereby, the noise-component detecting accuracy can be improved by emphasizing biological information. By performing filter processing for the acquired time-series data to remove low-frequency components including noise components, a waveform including biological information from which noise components have been removed is generated. The noise component includes components such as facial motions of the target to be inspected. As filter processing for removing low-frequency components, signal components in a specific frequency range are extracted by Bandpass Filter (BPF) processing. The waveform may be smoothed by performing moving average processing or noise removal processing that reduces noise components for the generated waveform. The method and range are not limited to those described above, and any method and range may be used.

For example, high-pass filter (HPF) processing may be used. Referring now to FIGS. 5A and 5B, a description will be given of one implementation. FIGS. 5A and 5B explain time-series data, and illustrate the time-series acquisition results of average luminance value signal data of the G signal within the target region. In each of FIGS. 5A and 5B, the vertical axis represents Gave (average luminance value), and the horizontal axis represents the number of frames. FIG. 5A illustrates illustrative time-series data expressed as Gave in a hand region of a target to be inspected, while most of the face region of the target to be inspected is hidden due to motions near the target to be inspected. Noise components are removed from the time-series data by BPF processing.

FIG. 5B illustrates illustrative time-series data expressed as Gave in the face region of a target to be inspected, while most of the face region of the target to be inspected is hidden by motions near the target to be inspected. Noise components are removed from the time-series data by BPF processing.

Although biological information can be obtained with high accuracy from the time-series data illustrated in FIG. 5A, it is difficult to obtain biological information from the time-series data illustrated in FIG. 5B. In a case where FIGS. 5A and 5B are compared, it can be understood that the amplitude change level in FIG. 5A is small, but the amplitude change level in FIG. 5B is large. In other words, by setting the body region in which time-series data has the smallest amplitude change level as the target region, a pulse rate as biological information can be acquired with high accuracy and stability. Thus, since time-series data including biological information is directly evaluated, a body region including biological information can be more accurately selected. The target region setting unit 14 compares amplitude changes in time-series data consisting of the total number of pixels, which is body region information acquired from each body region. The body region with the smallest amplitude change level in time-series data is determined to be the target region.

Thus, the target region setting unit 14 sets, as a single body region (target region), one of the plurality of body regions which has the smallest amplitude change level in average luminance value in time-series data regarding each of the plurality of body regions. In this example, for instance, a difference between the minimum value and the maximum value of the sampling values within a certain period (such as 10 seconds) can be used as the amplitude change, but the amplitude change is not limited to this implementation.

Next, in step S107 (biological information acquiring step) in FIG. 3, the biological information acquiring unit 15 acquires biological information. Blood pressure is estimated from the acquired waveform feature amount. It is known that blood pressure can be estimated from the outline and feature points of a pulse wave generated from a single target region. Therefore, in detecting the pulse wave, the blood pressure estimating accuracy changes depending on whether the waveform is in a state where noise can be sufficiently suppressed. Therefore, by removing the noise components using the filter processing for removing the low-frequency components, the feature amount can be accurately extracted from the generated waveform. In this example, the blood pressure estimating method is not limited to the method described above, and any method may be used.

As described above, in a case where event information is detected, the biological information detecting apparatus 10 according to this example can switch the target region based on the amplitude changes in the time-series data acquired from each of the body regions and thus can continuously detect biological information. Therefore, the biological information accuracy is improved. Furthermore, the biological information detecting apparatus 10 according to this example can select a body region containing biological information with higher accuracy than that in determining a body region to be a target region based on the total number of pixels as in Example 1.

### Example 3

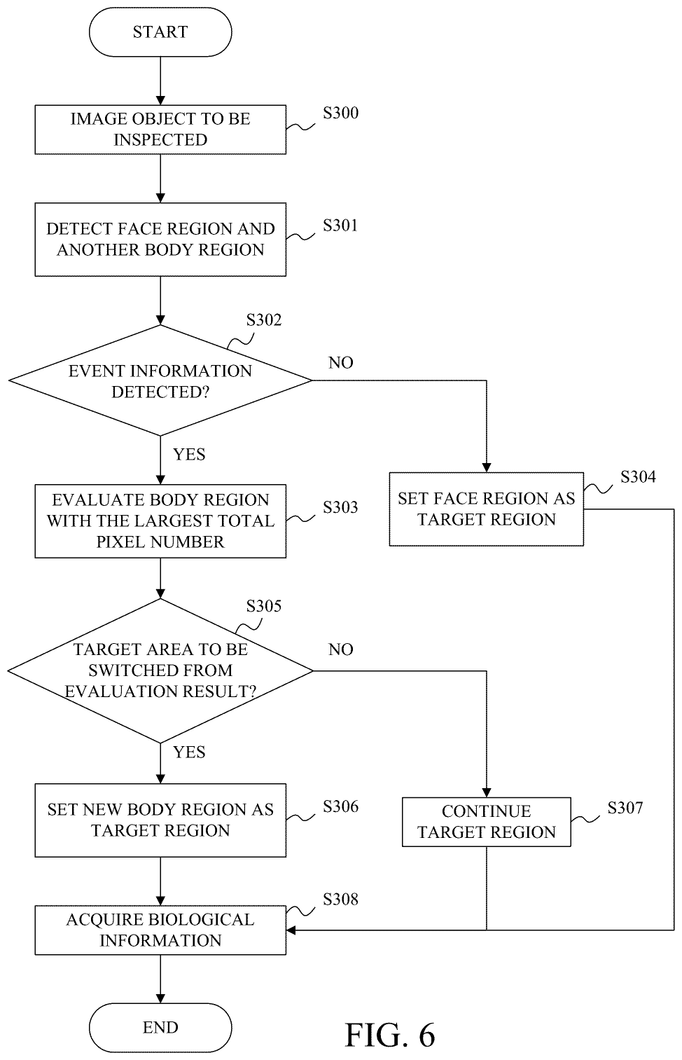

Referring now to FIG. 6, a description will be given of a detecting method (information processing method) by a biological information detecting apparatus 10 according to Example 3. FIG. 6 is a flowchart illustrating biological information detecting processing according to this example. In this example, the configuration of the image pickup apparatus 100 is similar to that of Example 1, and thus a description thereof will be omitted. In this example, steps S300, S303, S305, S306, and S307 in FIG. 6 are similar to steps S100, S103, S104, S105, and S106 in Example 1 (FIG. 3), and thus a description thereof will be omitted.

In step S301 (body region detecting step), the body region detector 12 detects at least two body regions including a face region including some of the facial parts such as the eyes, nose, mouth, cheeks, forehead, and facial outline of a target to be inspected, and another body region such as a hand region and a foot region.

Next, in step S302 (event information detecting step), the event information detector 13 determines whether or not event information has been detected. In this example, the event information is an illumination change. As the illumination changes, a luminance value of each pixel in a range corresponding to a part of the body region changes. In other words, in a case where the illumination environment changes, the condition for extracting the skin region changes, and consequently the total number of pixels that fill the skin region decreases and thereby the signal intensity of time-series data containing biological information decreases. In addition, in a case where the luminance value of each pixel in a range corresponding to a certain body region increases, a peak position cannot be accurately calculated from time-series data as a periodic light amount change of biological information on the target to be inspected. In other words, the biological information acquiring accuracy is affected. In a case where the event information detector 13 determines that event information has been detected, the flow proceeds to step S303. In step S303, the event information detector 13 compares the total number of pixels as body region information acquired from the body regions. On the other hand, in a case where the event information detector 13 determines that no event information has been detected, the flow proceeds to step S304.

In step S304 (target region setting step based on priority), the target region setting unit 14 sets a target region to a body region with higher priority, which is predetermined from among the body regions detected by the body region detector 12. For example, based on the total number of pixels of the body region, the priority has been determined to be higher in order of a face region, a hand region, and a foot region as a body region where biological information on the object can be acquired with high accuracy, and a target region from which the body region with higher priority can be acquired may be selected. In a case where the event information detector 13 has not detected event information, the biological information acquiring accuracy can be improved by preferentially selecting a body region in which biological information can be obtained with high accuracy.

In step S308 (biological information acquiring step), the biological information acquiring unit 15 acquires biological information. The respiration rate is calculated from the peak intervals in the waveform, which are time-series data generated by the biological information acquiring unit 15. The peak frequency may be calculated as a respiratory rate component from the frequency analysis result excluding noise components.

In a case where event information is detected, the biological information detecting apparatus 10 according to this example can switch the target region based on the total number of pixels acquired from each body region and thus continuously detect biological information. Furthermore, the biological information detecting apparatus 10 according to this example can preferentially select a body region as a target region in a case where no event information is detected. In addition, the biological information detecting apparatus 10 according to this example can achieve high detecting accuracy under various illumination changes.

### Example 4

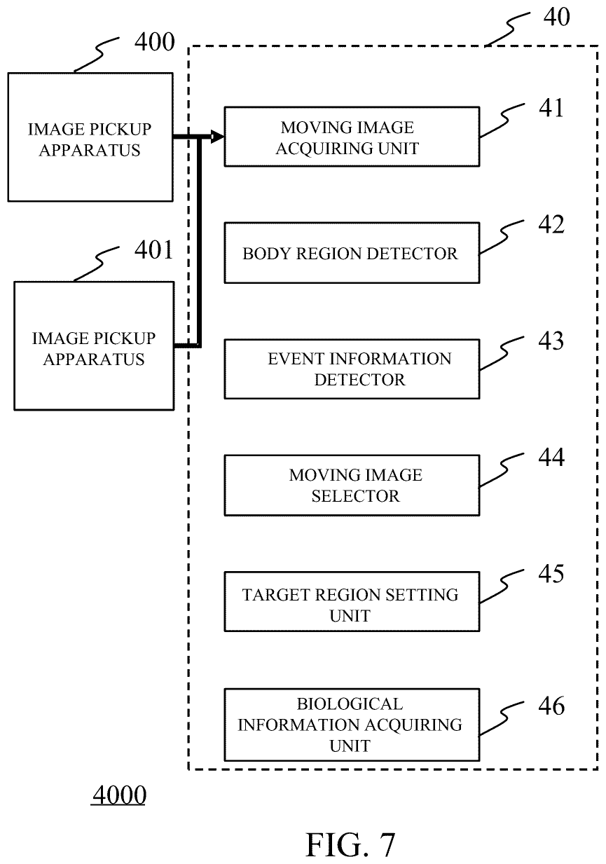

Referring now to FIG. 7, a description will be given of a detecting system (information processing system) 4000 including a biological information detecting apparatus (information processing apparatus) 40 according to Example 4. FIG. 7 is a block diagram of the detecting system 4000. The detecting system 4000 includes the biological information detecting apparatus 40 and two image pickup apparatuses 400 and 401. The biological information detecting apparatus 40 includes a moving image acquiring unit 41, a body region detector (region detector) 42, an event information detector (information detector) 43, a moving image selector 44, a target region setting unit (region setting unit) 45, and a biological information acquiring unit (information acquiring unit) 46. In this example, the body region detector 42, event information acquiring unit 43, target region setting unit 45, and biological information acquiring unit 46 in the biological information detecting apparatus 40 are similar to those of the biological information detecting apparatus 10 according to Example 1, and thus a description thereof will be omitted.

In this example, the image sensors included in each of the image pickup apparatus 400 and the image pickup apparatus 401 are RGB+IR sensors. The RGB+IR sensor has a G pixel that is sensitive to the G wavelength band, an R pixel that is sensitive to the R wavelength band, a B pixel that is sensitive to the B wavelength band, and an IR pixel that is sensitive to the IR wavelength band.

In this example, since the image sensor is an RGB+IR sensor, the moving image acquiring unit 41 acquires a G luminance value, an R luminance value, a B luminance value, and an IR luminance value. The image pickup apparatus 400 and the image pickup apparatus 401 temporally continuously images a target to be inspected at mutually different angles of view. Therefore, the moving image acquiring unit 41 can acquire luminance values corresponding to temporally continuous images with different viewing angles. The number of moving images with different angles of view that the moving image acquiring unit 41 acquires is not limited to two. The moving image selector 44 selects a moving image including a body region to be set as a target region from among the plurality of moving images with different angles of view acquired by the moving image acquiring unit 41.

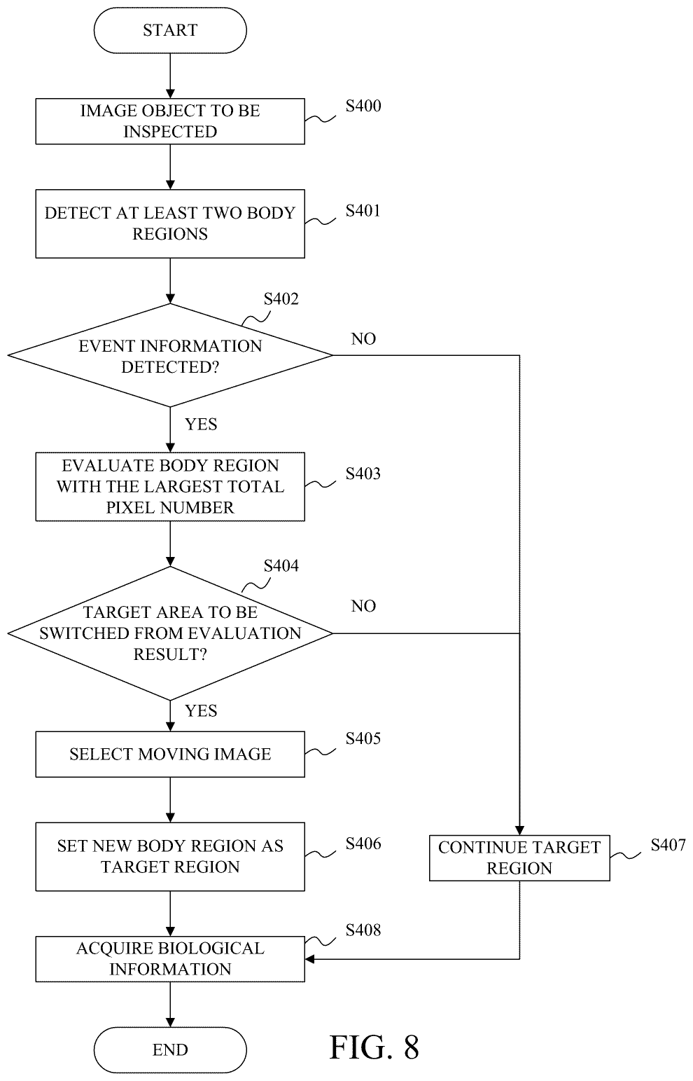

Referring now to FIG. 8, a detecting method (biological information detecting processing) by the biological information detecting apparatus 40 according to Example 4. FIG. 8 is a flowchart illustrating biological information detecting processing in this example. Steps S401, S402, S403, S404, S406, and S407 in FIG. 8 are similar to S101, S102, S103, S104, S105, and S106 in Example 1 (FIG. 3), and thus a description thereof will be omitted.

In step S400 (imaging step), the image pickup apparatuses 400 and 401 image a plurality of moving images with different angles of view including the target to be inspected.

In step S405 (moving image selecting step), the moving image selector 44 selects a moving image including a body region to be set as a target region from among the plurality of moving images with different angles of view acquired by the moving image acquiring unit 41.

In step S408 (biological information acquiring step), the biological information acquiring unit 46 acquires biological information. The biological information acquiring unit 46 calculates the pulse from the peak interval of the generated waveform. The biological information acquiring unit 46 also estimates the oxygen saturation level from calculations of the plurality of acquired waveforms.

As described above, in a case where event information is detected, the biological information detecting apparatus 40 according to this example can switch the target region based on the total number of pixels acquired from each body region. Furthermore, since the biological information detecting apparatus 40 according to this example can preferentially select a body region as a target region from a plurality of moving images with different viewing angles, a body region with higher signal intensity of biological information can be selected. In other words, high detecting accuracy can be achieved.

### Other Embodiments

Embodiment(s) of the disclosure can also be realized by a computer of a system or apparatus that reads out and executes computer-executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer-executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer-executable instructions. The computer-executable instructions may be provided to the computer, for example, from a network or the storage medium. The storage medium may include, for example, one or more of a hard disk, a random-access memory (RAM), a read-only memory (ROM), a storage of distributed computing systems, an optical disc (such as a compact disc (CD), digital versatile disc (DVD), or Blu-ray Disc (BD)™), a flash memory device, a memory card, and the like.

While the disclosure has described example embodiments, it is to be understood that some embodiments are not limited to the disclosed embodiments. The scope of the following claims is to be accorded the broadest interpretation so as to encompass all such modifications and equivalent structures and functions.

Each example can provide an information processing apparatus that can properly detect biological information on a target to be inspected.

This application claims priority to Japanese Patent Application No. 2023-105286, which was filed on Jun. 27, 2023, and which is hereby incorporated by reference herein in its entirety.

What is claimed is:

1. An information processing apparatus comprising:
a memory storing instructions; and
a processor configured to execute the instructions to:
acquire a moving image of a target to be inspected,
detect a plurality of body regions of the target to be inspected, from the moving image,
detect event information regarding a change in an object in the moving image,
set one body region selected from the plurality of body regions based on the event information, and
acquire biological information on the target to be inspected, from the set body region.

2. The information processing apparatus according to claim 1, wherein the processor is configured to:
evaluate the event information; and
determine whether to switch the one body region for acquiring the biological information among the plurality of body regions.

3. The information processing apparatus according to claim 1, wherein the event information is information regarding a motion of the object.

4. The information processing apparatus according to claim 3, wherein the object is the target to be inspected, a person different from the target to be inspected, and a thing in the moving image.

5. The information processing apparatus according to claim 1, wherein the event information is information regarding an illumination change.

6. The information processing apparatus according to claim 1, wherein the plurality of body regions include at least one of a face region, a hand region, and a foot region of the target to be inspected.

7. The information processing apparatus according to claim 1, wherein each of the plurality of body regions corresponds to a skin region of the target to be inspected.

8. The information processing apparatus according to claim 1, wherein the processor is configured to set a region having the largest total number of pixels in the moving image among the plurality of body regions as the one body region.

9. The information processing apparatus according to claim 1, wherein the processor is configured to set as the one body region, among the plurality of body regions, a region having the smallest amplitude change level in an average luminance value in time-series data regarding each of the plurality of body regions.

10. The information processing apparatus according to claim 9, wherein the time-series data is acquired by performing filter processing for removing low-frequency components.

11. The information processing apparatus according to claim 1, wherein in a case where the processor has not detected the event information, the processor is configured to set as the one body region a region set to highest priority among the plurality of body regions.

12. The information processing apparatus according to claim 11, wherein the plurality of body regions include at least one of a face region, a hand region, and a foot region of the target to be inspected,
wherein the priority is higher in order of the face region, the hand region, and the foot region.

13. The information processing apparatus according to claim 1, wherein the processor is configured to select the moving image from a first moving image and a second moving image that have different angles of view.

14. The information processing apparatus according to claim 1, wherein the biological information includes information regarding at least one of a pulse rate, a respiratory rate, blood pressure, or an oxygen saturation level of the target to be inspected.

15. An information processing system comprising:
an information processing apparatus; and
an image pickup apparatus,
wherein the information processing apparatus includes:
a memory storing instructions; and
a processor configured to execute the instructions to:
acquire a moving image of a target to be inspected,
detect a plurality of body regions of the target to be inspected, from the moving image,
detect event information regarding a change in an object in the moving image,
set one body region selected from the plurality of body regions based on the event information, and
acquire biological information on the target to be inspected, from the set body region.

16. An information processing method comprising the steps of:
acquiring a moving image of a target to be inspected, from an image pickup apparatus;
detecting a plurality of body regions of the target to be inspected;
detecting event information regarding a change in an object in the moving image;
setting one body region selected from the plurality of body regions based on the event information; and
acquiring biological information on the target to be inspected, from the set body region.

17. A non-transitory computer-readable storage medium storing a program that causes a computer to execute the information processing method according to claim 16.
